(19) United States
(12) Patent Application Publication
(10) Pub. No.: US 2004/0247563 A1
(43) Pub. Date: Dec. 9, 2004

(54) Method of enhancing lymphocyte-mediated immune responses

(76) Inventors: David H. Lynch, Seattle, WA (US); Thibaut N. De Smedt, Seattle, WA (US); Charles R. Maliszewski, Seattle, WA (US); Eric A. Butz, Seattle, WA (US); Robert E. Miller, Seattle, WA (US)

Correspondence Address: IMMUNEX CORPORATION, LAW DEPARTMENT, 1201 AMGEN COURT WEST, SEATTLE, WA, 98119, US

(21) Appl. No.: 10/399,116

(22) PCT Filed: Oct. 30, 2001
(86) PCT No.: PCT/US01/44834

Related U.S. Application Data
(60) Provisional application No. 60/245,721, filed on Nov. 2, 2000.

(51) Int. Cl. A61K 38/20

(52) U.S. Cl. 424/85.2; 514/12

(57) ABSTRACT

An improved method for treatment of an individual suffering from or at risk for an infectious disease, comprising administering to said individual a combination of from two to five agents is disclosed. The agents may be agents that mobilize dendritic cells, agents that cause death or growth inhibition of infectious agents, chemoattractants, agents that stimulate maturation of dendritic cells, and agents that enhance an immune response of an effector T cell. Antigen-expressing, activated dendritic cells are disclosed. Such dendritic cells are used to present antigens (specifically, antigens derived from infectious agents) to T cells, and can be useful in vaccination protocols. Useful cytokines can be used in separate sequential or simultaneous combination with the activated, antigen-pulsed dendritic cells. Also disclosed are methods for stimulating an immune response using the antigen-expressing, activated dendritic cells.

### FIELD OF THE INVENTION

The present invention relates to methods of enhancing a lymphocyte-mediated immune response, and to dendritic cell populations useful in the manipulation of cellular immune responses.

### BACKGROUND OF THE INVENTION

An immune response to a pathogen can be classified broadly as either being cell-mediated (cellular immunity) or antibody mediated (humoral immunity). In cellular immunity, activated macrophages and cytotoxic lymphocytes carry out elimination of the pathogen. Humoral immunity, in contrast, operates primarily through antibody production. It is currently believed that these two arms of the immune response are regulated by distinct subsets of helper T (Tₕ) cells which secrete specific arrays of cytokines.

Induction of cell-mediated immune responses requires the interaction of at least three different types of cells: dendritic cells (DC), CD4+helper T cells and CD8+effector T cells. Dendritic cells are a heterogeneous population of cells with a distinctive morphology and widespread tissue distribution. They are referred to as “professional” antigen presenting cells and have a high capacity for sensitizing MHC-restricted T cells. Tₕ cells generally fall into two categories: Type 1 (Tₕ1 cells) secrete Interferon-γ (IFN-gamma) and Interleukin-2 (IL-2) and stimulate development of cell-mediated immunity; and Type 2 (Tₕ2) cells secrete primarily Interleukins 4, 5, 10, and 13 (IL-4, IL-5, IL-10, and IL-13, respectively) and promote production of antibody by antigen-specific B cells.

Successful resolution of numerous infectious diseases depends on the nature of the immune response generated against the causative agent. For example, long-term survival of HIV infection appears to be associated with a strong cell-mediated (Tₕ2) immune response, whereas development of humoral immunity (Tₕ1 immune response) correlates with poor survival. Thus, there is a need in the art for treatments that promote generation of primarily cell-mediated immune responses, in particular to HIV.

### SUMMARY OF THE INVENTION

The present invention provides methods for treating an individual afflicted with, or at risk for, a condition characterized by the presence of a pathogenic or opportunistic organism (or two or more such organisms), comprising the steps of: (a) administering a DC mobilization factor; and (b) administering an agent that enhances a cytotoxic T lymphocyte response against the pathogenic or opportunistic organism. In one aspect of the invention, the pathogenic or opportunistic organism is HIV; in a preferred embodiment, the inventive method is used in conjunction with HAART. In yet another aspect of the invention, an agent that stimulates maturation of DC is administered to the individual. The methods described herein optionally further include the steps of administering a chemoattractant to attract mobilized dendritic cells and/or T cells to a specific site, such as a lymph node. Optionally, the methods may further include administering antigen(s) to the individual.

In one embodiment, the methods of the present invention are in vivo combination immunotherapy methods in which the just described agents (DC mobilization factor, DC maturation agent, T lymphocyte enhancing agent, and chemoattractant) are administered to the individual by any suitable method, including topically, subcutaneous, intravenous, intranodal or intramuscular administration, administration in the form of a controlled or sustained release formulation, oral administration, or use of any other route known to one of routine skill in the art. Moreover, the various agents may be administered locally, in or near a site of infection, for example by application of a localized sustained release formulation during or immediately after surgery or other treatment, or by use of other methods known in the art to deliver an agent or agents to a specific site.

In another embodiment, the methods of the present invention are combination immunotherapy methods in which one or more of the above described administering steps is performed ex vivo. For example, the present invention provides combination therapies that include (a) administering a therapeutically effective amount of a DC mobilization factor to a an individual afflicted with a condition characterized by the presence of a pathogenic or opportunistic organism; (b) obtaining dendritic cells from the individual; (c) culturing the dendritic cells obtained from the individual in an ex vivo culture; and (d) administering the cultured dendritic cells to the individual.

Optionally, the ex vivo combination immunotherapy methods of the present invention further include the step of contacting the cultured dendritic cells with an antigen in such a way that the cells are able to present the antigen to other immune cells. Additionally the ex vivo methods may include the step of treating cultured dendritic cells with an agent that stimulates activation and/or maturation of dendritic cells in order to facilitate antigen presentation. The step of treating the cultured dendritic cells with an agent that stimulates activation and/or maturation of dendritic cells may be performed before or after contacting the cultured dendritic cells with the antigen, depending upon whether the antigen requires processing or not. Typically, if the antigen requires processing by the dendritic cell, treating the cultured dendritic cells is performed after the dendritic cells have processed the antigen. If the antigen does not require processing by the cultured dendritic cells, treating the cultured dendritic cells with an agent that stimulates activation and/or maturation of dendritic cells step is performed prior to (or concurrently with) contacting the cultured dendritic cells with antigen.

In yet another embodiment, the present invention further includes causing the dendritic cells to secrete certain cytokines. In ex vivo methods, this may be accomplished by contacting the dendritic cells with one or more agents that induce the cytokine expression, or by transfecting dendritic cells with a gene(s) encoding a cytokine(s).

Concurrent with administering cultured DC to an individual afflicted with a condition characterized by the presence of a pathogenic or opportunistic organism, the present invention further includes administering cultured DC or mature, antigen-presenting DC alone or in combination with T cell enhancing agent(s). In an alternative approach, the methods of the invention include generating antigen-specific T cells ex vivo using the cultured DC and administering the antigen-specific T cells to the individual. A T cell enhancing agent may be administered to the individual prior to obtaining T cells; alternatively or additionally, a T cell enhancing agent may be administered to the individual in conjunction with ex vivo-generated antigen-specific T cells.

The methods of the present invention further include administering a chemoattractant to attract mobilized DC and/or T cells, NK cells or other immune cells to a specific site (i.e., attracting antigen-carrying DC to a T cell-rich lymph node or attracting immune cells to a site of infection).

Combination immunotherapy methods described herein are useful in treating individuals suffering from immunosuppression that can occur in individuals infected with a pathogenic or opportunistic organism, since many pathogens have immunosuppressive effects. The immunotherapy methods of the invention stimulate an immune response and facilitate recovery of the immune system from the side effects of the infection.

Many DC mobilization factors enhance the population of bone marrow progenitor cells in the infected individual. If desired, the inventive methods may be used as part of an immunization regimen to generate an effective immune response against a desired antigen in the individual afflicted with a condition characterized by the presence of a pathogenic or opportunistic organism.

The inventive methods may be used to generate or regenerate an immune response in the individual afflicted with, or at risk for, a condition characterized by the presence of a pathogenic or opportunistic organism ex vivo by: (a) administering a therapeutically effective amount of a DC mobilization factor to the individual; (b) obtaining dendritic cells from the individual; (c) culturing the dendritic cells ex vivo; and (d) administering the dendritic cells to the individual.

In yet another aspect of the instant ex vivo therapy, the dendritic cells are treated with an antigen against which it is desired to generate an immune response. The dendritic cells may also be caused to secrete certain desirable immunologically active agents; they may be administered alone or in combination with agents that enhance a cytotoxic T lymphocyte or helper cell response against the antigen, or a T cell growth factor to stimulate proliferation of T cells. Alternatively, the dendritic cells may be used to generate antigen-specific cytotoxic T cells or helper cells ex vivo, which are then administered to the individual. These and other aspects of the invention will be apparent to one of ordinary skill in the art.

The activated antigen-presenting dendritic cells can also be used as a vaccine adjuvant and can be administered prior to, concurrently with or subsequent to administration of an antigen. Moreover, the dendritic cells can be administered to the individual prior to, concurrently with or subsequent to administration of cytokines that modulate an immune response, in particular, agents that enhance a cytotoxic T lymphocyte response against the antigen.

The invention also provides for the ex vivo preparation of antigen-specific (for example, HIV-specific) T cells. Following the procedures described above for preparing large numbers of antigen-presenting dendritic cells ex vivo, the collected antigen-presenting dendritic cells are used to generate antigen-specific cytotoxic T cells ex vivo, which are then administered to the individual.

### BRIEF DESCRIPTION OF THE DRAWINGS

FIG. 1 is a flowchart depicting various steps in the inventive method(s). Those steps that must be performed in vivo are listed on the left side of the flow chart, while those that may be performed ex vivo are shown on the right side. While the steps are shown in the general order in which they would usually be performed, those of ordinary skill in the art are able to optimize the order and/or timing of the steps, as well as the dosages and routes of administration, by routine experimentation. Thus, for example, an anti-infective agent can be administered by any means disclosed herein; the optimal time to administer a dendritic cell (DC) maturation agent and/or cultured DC (either immature or activated, mature DC) will depend on the nature of the anti-infective agent and its effects, if any, on the DC. Similarly, as described in detail herein, when preparing mature, activated, antigen-carrying DC ex vivo, those of ordinary skill in the art will adjust the steps performed ex vivo to optimize activation and antigen presentation ability (i.e., generally, with peptide antigens, the DC are contacted with the peptide after maturation, whereas with larger antigens that require processing, the DC are usually contacted with the antigen and allowed to process it prior to maturation). Moreover, the skilled artisan can utilize chemoattraction to enhance trafficking of cells to a specific site by localized administration (achieved by any method described herein) of a chemokine or chemokine-inducing agent, for example, administering a chemokine (or chemokine inducer) that attracts DC to a site of infection to increase the numbers of DC that take up antigen, or administering a chemokine (or chemokine inducer) into a lymph node to facilitate trafficking of antigen-carrying DC to a T cell-rich area. Additionally, an agent that enhances the numbers of circulating T cells can be administered to the individual prior to obtaining T cells for ex vivo culture. The same agent (or another T cell enhancing agent) may be administered when expanded T cells are administered to the individual.

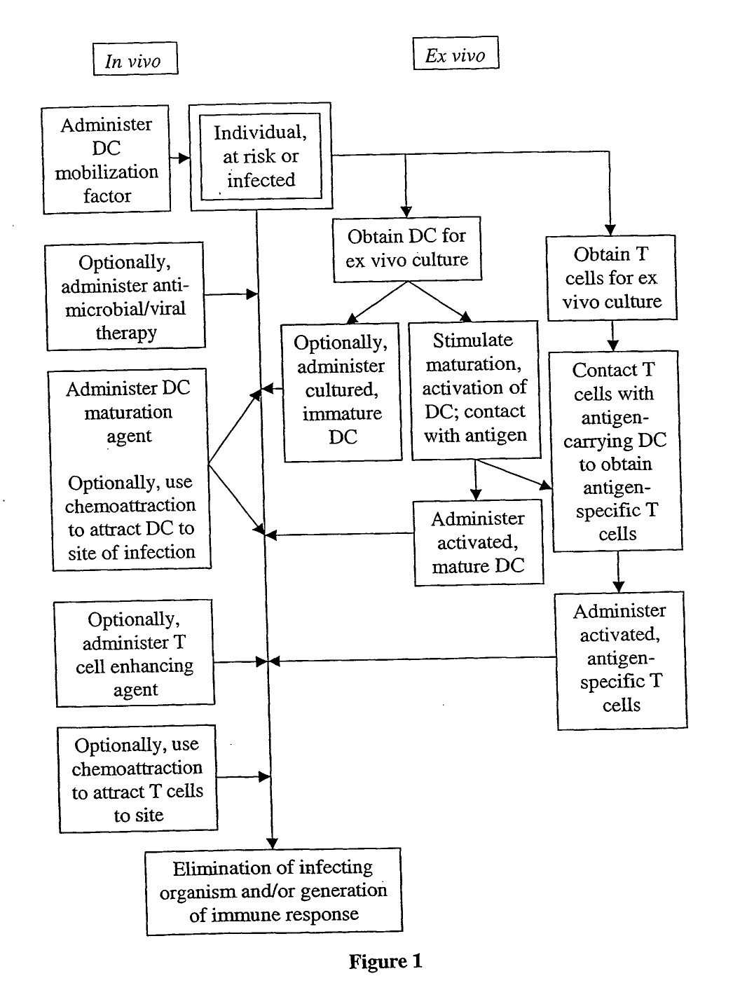

FIG. 2 presents the nucleotide and amino acid sequence of human granulocyte-macrophage colony stimulating factor.

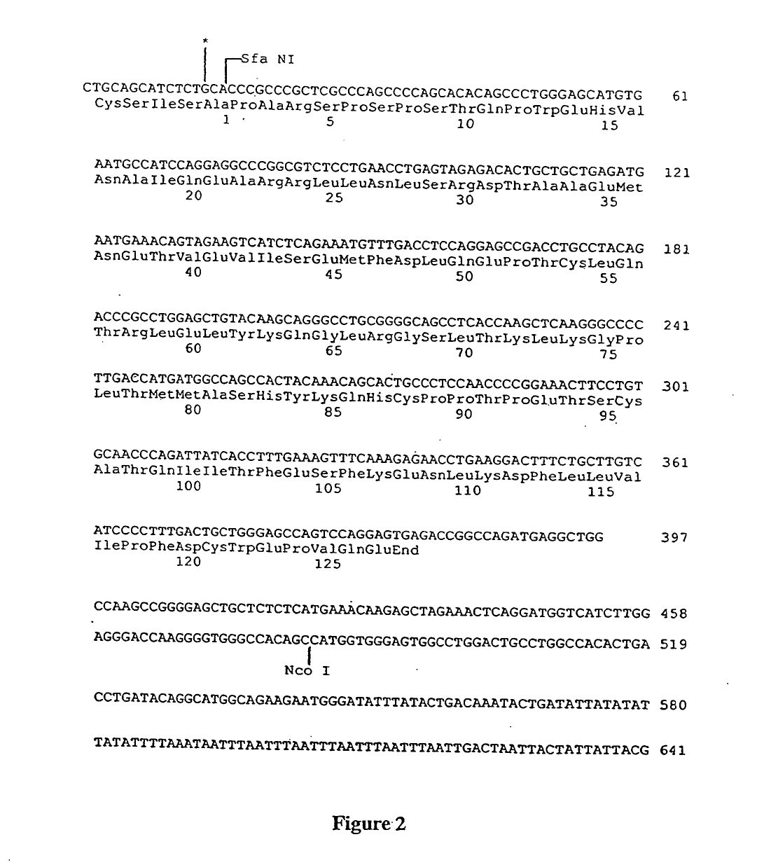

### DETAILED DESCRIPTION OF THE INVENTION

Administering an agent that increases the number of DC facilitates uptake and processing of antigen from a pathogenic or opportunistic organism. When contacted with a maturation factor, the DC induce a potent memory or primary CTL response specific to the organism. T cell growth factors (including growth and/or survival factors as well as co-stimulatory factors, either endogenously provided by activated DC or exogenously added) will further expand the antigen-specific CD4+ and CD8+T cell populations, which then act to eradicate the pathogenic or opportunistic organism in infected individuals, or act to prevent infection in individuals at risk for infection.

The present invention will also be useful in facilitating recovery of individuals from immunosuppression that occurs as a result of the presence of a pathogenic or opportunistic organism. An agent that increases the number of DC may be administered, and the DC obtained and preserved for subsequent re-administration to the individual. The DC may be treated ex vivo to allow them to more effectively present antigen to other immune cells; moreover, ex vivo techniques can also be applied to obtain antigen-specific effector cells such as cytotoxic T cells specific for a particular pathogenic or opportunistic organism.

Pathogenic/Opportunistic Organisms

Pathogenic organisms are organisms that are capable of causing disease in a healthy individual, whereas opportunistic organisms usually do not cause disease in a healthy individual, but may result in disease conditions in immunocompromised hosts. Both types of organisms include viruses, bacteria, yeast, fungi, and protozoa. Additionally, in some syndromes, multiple organisms may be present, and may play a causative role in the syndrome.

An exemplary pathogenic protozoan is Leishamania, an obligate intracellular macrophage parasite that causes a variety of diseases characterized by visceral, cutaneous, or mucosal lesions. Different species and isolates of Leishmania vary in their ability to infect and replicate in macrophages both in vivo and in vitro. Clinically, infections with L. braziliensis present as single or multiple cutaneous lesions, with a small percentage progressing to a more severe mucosal disease. While the cutaneous lesions may heal spontaneously or respond well to chemotherapy, mucosal lesions are often highly destructive and relatively refractory to treatment. Even if the mucosal lesion cures, there is often spontaneous relapse, perhaps years later.

The pathogenic hemoflagellate protozoan Trypanosoma cruzi (T. cruzi) causes Chagas' disease, a major public health problem in many countries of Latin America. Infection with this parasite may be acute or chronic, and frequently involves development of progressive pathology in tissues of the heart, esophagus and colon. The parasites infect a variety of nucleated cells, including macrophages. In both humans and laboratory animals, T. cruzi infection is accompanied by a non-specific immune-suppression mediated by T cells and macrophages. Mechanisms which control parasite replication during the acute and chronic phases, and which maintain low but persistent numbers of circulating parasites during the chronic phase, are not well understood.

Additional examples of pathogens include Mycobacterium tuberculosis, Mycobacterium avium complex, and Mycobacterium leprae, as well as the protozoan Toxoplasma gondii. The fungi Histoplasma capsulatum, Candida albicans, Candida parapsilosis, and Cryptococcus neoformans can also be considered opportunistic or pathogenic organisms. Certain of the Rickettsia, for example, R. prowazekii, R. coronii, and R. tsutsugamushi are also included, as are combinations of two or more organisms.

In addition to infecting humans, many of these organisms infect other mammals, which then can serve as a reservoir of infection for humans. For example, domesticated dogs are believed to serve as a major reservoir of Leishmania, while cats are known to carry Toxoplasma. Methods of augmenting a mammal's immune and/or inflammatory response against these organisms are thus likely to be useful in species of mammals other than humans.

Numerous viruses are known to infect humans. Among them are members of Herpesviridae, a family of DNA-containing viruses that includes the viruses that cause chickenpox (Herpes zoster, which also plays a role in shingles), oral and genital herpes (Herpes simplex), and mononucleosis (Epstein-Barr virus). Cytomegalovirus (CMV) is another member of this family; infection is widespread but does not usually result in illness in an immunocompetent individual. However, CMV (and other herpes viruses) can cause clinical disease in immunocompromised individuals such as organ transplant recipients, individuals undergoing immunosuppressive therapy, and people afflicted with acquired immunodeficiency syndrome (AIDS).

Progressive multifocal leukoencephalopathy is a demyelinating disease that occurs in immunosuppressed hosts; it is caused by a papovavirus, a member of a group of DNA viruses that includes papilloma viruses (implicated in warts and some cancerous or precancerous conditions) and polyoma viruses. Several different viruses cause hepatitis, a syndrome characterized by inflammation of the liver. The are generally referred to as Hepatitis A virus, hepatitis B virus, etc.; currently, there are five known, distinct hepatitis viruses, which are related by the disease they cause, not by type of genome, genetic similarity, or other parameters used to classify viruses into groups.

Paramyxoviruses are single-stranded RNA viruses that cause numerous diseases, including Newcastle disease, measles, and subacute sclerosing panencephalitis. One member of this family, respiratory syncytial virus (RSV) causes a cold-like respiratory infection in young children that can be especially problematic in infants. Influenza is an acute viral infection of the respiratory tract that can occur sporadically or in epidemics or pandemics. It is particularly dangerous for individuals who have a diminished immune system, including the elderly and the very young. Influenza is caused by a number of serologically distinct strains of Orthomyxoviruses, designated A (with many subgroups), B and C.

Other RNA viruses are also the causative agents of human disease. Reovirus 3 has been implicated in biliary atresia and neonatal hepatitis, although other members of the reovirus family do not appear to cause disease. However, the virus that causes Colorado Tick Fever has been classified as a reovirus, and Rotavirus (another member of the Reoviridae family) is thought to be the most important cause of severe dehydrating diarrhea in children under three years of age worldwide. Picorna viruses are single-stranded RNA-containing viruses that cause hepatitis in humans; this family includes poliovirus, the causative agent of poliomyelitis.

Many cancers in vertebrates are caused by retroviruses, a group of RNA viruses that uses the enzyme reverse transcriptase to copy its genome into the DNA of the host cell chromosome. Included in this family are human t-lymphotropic virus types I and II (HTLV I and HYLV II), as well as human immunodeficiency virus (HIV). HTLV I causes adult T-cell leukemia and T-cell lymphoma and may also be involved in certain demyelinating diseases; HTLV II may also be involved in these conditions. HIV is the causative agent of AIDS.

Another type of microorganism that causes infection that may be treated according to the inventive methods is Mycoplasma pneumoniae. Mycoplasma are prokaryotic microorganisms that lack cell walls and that therefor cannot be treated with certain antibiotics. Those of skill in the art are aware of the aforementioned agents, and of others that may be treated according to the inventive methods. More information on these and other infectious agents can be found in numerous, well-known resources, including Mandell, Douglas and Bennett's Principles and Practice of Infectious Diseases, 5^{th} edition (Mandell, Douglas, Bennett and Dolin, eds.; Churchill Livingstone, 2000; New York) and Mims' Pathogenesis of Infectious Disease. 5^{th} edition (Mims, Nash and Stephen, eds., Academic Press, Incorporated, 2000).

Various computer and Internet-based resources are available to assist the skilled artisan in diagnosing and treating infectious disease. An exemplary web site is that maintained by the Centers for Disease Control of the National Institutes of Health of the U.S. (http://www.cdc.gov/ncidod/). The website contains information about various types of infectious disease, treatment options, various clinical trials that are ongoing, risk factors in infectious disease, and other helpful resources.

Combination Therapy

A combination of from two to four components will be useful in the present immune-based anti-infective therapy model. Useful components include factors that increase the numbers of antigen-presenting cells (APC), especially DC (DC mobilization factors), and factors that lead to T helper cell expansion and activation (Th enhancing factors) and T effector cell expansion and immune activation (CTL enhancing factors). Additional useful factors include factors that stimulate the maturation of the APC or DC. Together, these are referred to as immunomodulatory agents. Moreover, the inventive methods may be used in conjunction with anti-viral, anti-microbial, or anti-infective therapy, including highly-active antiviral therapy (HART) for HIV infection. HAART involves treating an individual infected with HIV with a combination of anti-viral agents, usually targeted at different stages of viral replication, and has been found to decrease both morbidity and mortality in infected individuals (Palella et al., N. Engl. J. Med. 338:853, 1998; DeSimone et al., Ann. Internal Med. 133:447, 2000).

TRAIL refers to a genus of polypeptides that induce apoptosis of certain target cells, including virally-infected cells. The cloning and characterization of TRAIL is described in U.S. Pat. No. 5,763,223, issued Jun. 9, 1998. As disclosed therein, TRAIL comprises an N-terminal cytoplasmic domain, a transmembrane region and an extracellular domain. Soluble forms of TRAIL that are useful in the present invention include the extracellular domain of TRAIL or a fragment of the extracellular domain that retains the ability to bind to target cells and induce apoptosis. A preferred form of soluble TRAIL comprises amino acids 95 through 281 of human TRAIL (SEQ ID NO:5) as disclosed in U.S. Pat. No. 5,763,223.

Oligomeric forms of TRAIL are also useful; preferred forms comprise the extracellular domain of TRAIL fused to a peptide that facilitates trimerization. Peptides derived from naturally occurring trimeric proteins or synthetic peptides that promote oligomerization may be employed. Particularly useful peptides are those referred to as leucine zippers (zipper domains or leucine zipper moieties). In particular embodiments, leucine residues in a leucine zipper are replaced by isoleucine residues. Such peptides comprising isoleucine may be referred to as isoleucine zippers, but are encompassed by the term “leucine zippers” as employed herein.

One example is a leucine zipper derived from lung surfactant protein D (SPD), as described in Hoppe et al. (FEBS Letters 344:191, 1994) and in U.S. Pat. No. 5,716,805, comprising amino acids Pro Asp Val Ala Ser Leu Arg Gln Gln Val Glu Ala Leu Gln Gly Gln Val Gln His Leu Gln Ala Ala Phe Ser Gln. Another example of a leucine zipper that promotes trimerization is the zipper peptide shown in SEQ ID NO:4. In an alternative embodiment, the peptide lacks the N-terminal Arg residue. In another embodiment, an N-terminal Asp residue is added. Yet another example of a suitable leucine zipper peptide comprises the amino acid sequence Ser Leu Ala Ser Leu Arg Gln Gln Leu Glu Ala Leu Gln Gly Gln Leu Gln His Leu Gln Ala Ala Leu Ser Gln Leu Gly Glu. In an alternative peptide, the leucine residues in the foregoing sequence are replaced with isoleucine. Fragments of the foregoing zipper peptides that retain the property of promoting oligomerization may be employed as well. Examples of such fragments include, but are not limited to, peptides lacking one or two of the N-terminal or C-terminal residues presented in the foregoing amino acid sequences.

Factors that increase the number of DC when administered in vivo are referred to as DC mobilization factors (or agents). Suitable DC mobilization factors include, but are not limited to, Flt3L, granulocyte-macrophage colony stimulating factor (GM-CSF), granulocyte colony stimulating factor (G-CSF), CD40L and Interleukin-15 (L-15). Different DC mobilization factors mobilize distinct subsets of DC in humans. Flt3L increases both CD11c+ and CD11c-IL-3R+subsets; the former subset is increased between 40- and 50-fold and the latter is increased between 10- and 15-fold (Pulendran et al., J. Immunol. 165:566, 2000; Maraskovsky et al., Blood 96:878, 2000). In contrast, G-CSF increases only the CD11c−subset, and that by about 7-fold (Pulendran et al., supra). Because the two subsets of DC elicit different cytokine profiles in CD4+ T cells, different DC mobilization factors may be used to preferentially enhance one type of immune response over another (i.e., Tₕ1-like response versus Tₕ2-like response).

Flt3L refers to polypeptides that bind the cell-surface tyrosine kinase receptor Flt3, and regulate the growth and differentiation of progenitor and stem cells thereby. U.S. Pat. No. 5,554,512, issued Sep. 10, 1996 (herein incorporated by reference), describes the isolation of a cDNA encoding Flt3L, and the use of this molecule in peripheral stem cell transplantation procedures. Various forms of Flt3L are described therein, including both human and murine Flt3L, fusion proteins and muteins. Preferred Flt3L polypeptides comprise amino acids 28 through 160, amino acids 28 through 182, or amino acids 28 through 235 of human Flt3L (SEQ ID NO:2), and fragments thereof. Particularly preferred Flt3L polypeptides comprise amino acids 28 through 179 or amino acids 26 through 179 of SEQ ID NO:1.

Other Flt3L related dendritic cell mobilization agents suitable for use in the present invention include those agents that bind Flt3 and transduce a signal. Such Flt3 binding proteins encompass agonistic antibodies that include monoclonal antibodies and humanized antibodies, and recombinantly-prepared agents that have at least one suitable antigen binding domain and are derived from agonistic antibodies that transduce Flt3 signaling.

GM-CSF is a lymphokine that induces the proliferation and differentiation of precursor cells into granulocytes, macrophages, and DC. U.S. Pat. No. 5,162,111, issued Nov. 10, 1992, discloses the nucleotide and amino acid sequence of both human and murine GM-CSF, and describes the use of this lymphokine in treating bacterial diseases. Other forms of GM-CSF will also be useful in the instant invention, including fusion proteins comprising GM-CSF and Interleukin-3 (described in U.S. Pat. No. 5,108,910, issued Apr. 28, 1992), muteins of GM-CSF (disclosed in U.S. Pat. No. 5,391,485, issued Feb. 21, 1995), and prolonged-release compositions comprising GM-CSF (described in U.S. Pat. No. 5,942,253, issued Aug. 24, 1999). The relevant disclosures of the above-referenced patents are specifically incorporated herein.

IL-15 is a secreted cytokine that is produced as a precursor protein and cleaved to its active form. Mature IL-15 is capable of signaling the proliferation and/or differentiation of precursor or mature T-cells as well as maintaining memory T cells, and so can be used (in vivo or ex vivo) to regulate a T cell immune response. IL-15, which has been referred to as Epithelium-derived T-Cell Factor is described in U.S. Pat. No. 5,574,138, issued Nov. 12, 1996 (incorporated herein by reference). Preferred forms of IL-15 comprise mature IL-15 polypeptides (amino acids 49 through 162 of the non-cleaved precursor protein; SEQ ID NO:2).

DC can also be grown ex vivo after mobilization with Flt3L, GM-CSF, granulocyte colony stimulating factor (G-CSF), cyclophosphamide or other agents known to mobilize CD34+cells. The DC so obtained can be cultured using agents such as Flt3L, GM-CSF, Interleukin-15 (IL-15), CD40 Ligand (CD40L), TNF-α or the ligand for receptor activator of NF-kappaB (RANKL). Alternatively, DC can be generated from peripheral blood mononuclear cells (PBMC) using GM-CSF and Interelukin-4 (IL4). The DC generated ex vivo by these methods may be administered locally into a specific site (i.e., into a site of infection with a pathogen or opportunist), systemically into the bloodstream or into draining lymph nodes.

Suitable DC maturation agents useful in the practice of the invention include CD40L and agonists of CD40 signaling, RANKL, TNF, IL-1, CpG-rich DNA sequences (ISS, or immunostimulatory sequences), lipopolysaccharide (LPS), and monocyte-conditioned medium (Reddy et al., Blood 90:3640;1997). These factors act on DC by enhancing their capabilities to stimulate an effective, specific, cytotoxic immune response. Thus, for example, ligation of CD40 on DC by CD40L or another CD40 agonist stimulates an increase in the numbers of MHC molecules on the surface of the DC, which increases their antigen-presenting capacity. In addition, stimulation with maturation factors may also enhance secretion of various immunomodulatory cytokines (for example, IL-12) which can act to augment the immune response. DC may also be contacted with agents that stimulate secretion of cytokines that indicate that the DC are activated (DC activation factors). Thus, for example, DC may be contacted with CD40L and IFN-gamrna (simultaneously, sequentially or separately) to stimulate maturation and activation of DC.

CD40L polypeptides which are capable of binding CD40, and transducing a signal thereby, are useful in the present invention. cDNAs encoding CD40L are described in U.S. Pat. Nos. 5,961,974, 5,962,406 and 5,981,724 (hereinafter, the Armitage patents). The nucleotide sequence and amino acid sequence of representative murine and human CD40L cDNA is disclosed in the Armitage patents, and is hereby incorporated by reference. Forms of CD40L that are particularly useful maturation agents include the extracellular portion of CD40L and fragments of the extracellular portion that bind CD40 and transduce a signal. In particular, polypeptides that include amino acids 47-261 of SEQ ID NO:3, polypeptides that include amino acids 113-261 of SEQ ID NO:3, polypeptides that include amino acids 51-261 of SEQ ID NO:3 and oligomeric forms of these polypeptides, as disclosed in the Armitage patents, can be used in the present invention. A preferred CD40L is one in which the cysteine amino acid 194 of human CD40L is substituted with tryptophan. A most preferred form of CD40L is a soluble CD40L fusion protein referred to as trimeric CD40L in the Arnmitage patents. Trimeric CD40L comprises a fragment of the extracellular domain of CD40L fused to a zipper domain that facilitates trimerization (SEQ ID NO:4).

Additional suitable dendritic cell maturation agents include compounds that bind CD40 and transduce a signal. Amongst these are agonistic antibodies to CD40 such as monoclonal antibody HuCD40-M2 (ATCC HB 11459) as well as humanized antibodies or other, recombinantly-derived molecules comprising an antigen binding domain derived from antibody HuCD40M2.

RANKL, like CD40L, is a Type 2 transmembrane protein with an intracellular domain of less than about 50 amino acids, a transmembrane domain and an extracellular domain of from about 240 to 250 amino (SEQ ID NO:6). RANKL is described in U.S. Ser. No. 08/995,659, filed Dec. 22, 1997 (PCT/US97/23775). Similar to other members of the TNF family to which it belongs, RANKL has a spacer region between the transmembrane domain and the receptor binding domain that is not necessary for receptor binding. Accordingly, soluble forms of RANKL can comprise the entire extracellular domain or fragments thereof that include the receptor binding region.

Similarly to CD40L, other compounds that bind RANK and transduce a signal are useful maturation agents and include agonistic antibodies to RANK as well as humanized antibodies or other, recombinantly-derived molecules comprising an antigen binding domain derived from antibody that binds RANK. Several other members of the TNF superfamily will also have use in various aspects of the instant invention. These include lymphotoxins alpha and beta, Fas ligand, CD27 ligand, CD30 ligand, CD40 ligand, 4-1BB ligand, OX40 ligand, TRAIL and RANKL.

DC can also be grown ex vivo after mobilization with Flt3L, GM-CSF, granulocyte colony stimulating factor (G-CSF), cyclophosphamide or other agents known to mobilize CD34+cells. The DC so obtained can be cultured using agents such as Flt3L, GM-CSF, Interleukin-15 (IL-15), CD40 Ligand (CD40L) or the ligand for receptor activator of NF-kappaB (RANKL). Alternatively, DC can be generated from peripheral blood mononuclear cells (PBMC) using GM-CSF and Interleukin-4 (IL-4). Cultured DC can further be treated ex vivo to stimulate maturation and/or activation as described above. The DC generated ex vivo by these methods may be administered locally into a site of infection, systemically into the bloodstream or into draining lymph nodes.

TNF is a dendritic cell maturation agent that also plays a central role in inflammatory and immune defenses, and is involved in several pathogenic processes, including cachexia, septic shock and autoimmunity. Its potent effects on cells of the immune system render it useful in vitro (for example, in ex vivo generation, expansion and/or activation of cells, and/or maturation of DC). Moreover, various techniques can be used to minimize systemic effects, for example, use in gene therapy or local administration in or near the site of infection, as discussed herein.

Lipopolysaccharide (LPS), another dendritic cell maturation agent, is a component of the cell wall of Gram-negative bacteria. LPS consists of a lipid core (lipid A) and an attached polysaccharide moiety; the lipid A (along with some associated polysaccharides) is thought to be responsible for most of the toxic effects of Gram-negative bacteremia, including toxic shock syndrome (septic shock or endotoxemia). LPS may be used ex vivo to generate mature DC; alternatively, various techniques described herein can be applied to allow for localized administration of LPS to an infected individual.

Additional suitable dendritic cell maturation agents include those agents that are also suitable T-cell enhancing agents. Such agents include Interleukins 2, 15, 7 and 12, (IL-2, IL-15, IL-7, and IL-12, respectively) and interferons-gamma and -alpha (IFN-gamma and IFN-α), and OX40 and 4-1BB agonists. These agents, and many others that have utility in the present combination therapy method, are described in The Cytokine Handbook (third edition; edited by Angus Thompson; Academic Press 1998).

First identified as a T cell growth factor, Interleukin-2 (IL-2) is also known to affect B cells, natural killer (NK) cells, lymphokine-activated killer (LAK) cells, monocytes, macrophages and oligodendrocytes. The three-dimensional structure of this 15.5 kDa glycoprotein has been determined, and various types of studies have elucidated the function its various domains. IL-2 has been shown to have antitumor activity against some renal cell carcinomas and melanomas, however, toxic effects have limited its use as in single-agent immunotherapy. Nonetheless, because of its potent effects, there is continued interest in developing IL-2 formulations and/or methods of administration that will be tolerated by patients. U.S. Pat. No. 6,060,068, issued May 9, 1000, describes IL-2 and its use as a vaccine adjuvant; the use of IL-2 in gene therapy is described in U.S. Pat. No. 6,066,624, issued May 23, 2000. The use of IL-2 in conjunction with heat shock protein/antigenic peptide complexes for the prevention and treatment of neoplastic disease is described in U.S. Pat. No. 6,017,540, issued Jan. 25, 2000. Interleukin-7 (IL-7) is a cytokine of about 25 KDa that is secreted by both immune and non-immune cells, and is involved in the development of the immune systems and generation of a cellular immune response. U.S. Pat. No. 5,328,988, issued Jul. 12, 1994, describes the identification and isolation of human IL-7. Because IL-7 enhances the immune effector cell functions of T lymphocytes, it may be used in the inventive therapy as an agent that augments a CTL response. IL-7 also acts as a growth factor and has been used to stimulate the growth of immune cells after bone marrow transplantation or high-dose chemotherapy. Accordingly, IL-7 may also find use in the instant invention as an agent that mobilizes or stimulates the growth of immune cells.

Biologically active interleukin-12 (IL-12) is a heterodimeric protein consisting of a heavy chain (p40) that bears structural resemblance to the Interleukin-6 (IL-6) receptor and the G-CSF receptor, and a light chain (p35) that resembles IL-6 and G-CSF. Because of its ability to promote the preferential development of a Th1 immune response, IL-12 has been used in the infectious disease setting as well as in tumor models. Accordingly, IL-12 can be utilized in the combination therapy described herein to enhance CTL activity. Additionally, IL-12 may be used in a genetic therapy-based approach, either transducing target cells or DC to express IL-12, then administering the IL-12-expressing cell. When used in such modalities, and when administered systemically, IL-12 can induce target cell apoptosis, and thus may also be used in the instant invention as an apoptotic agent.

Interferons fall into two categories referred to as Type I interferons (IFN-α, IFN-ω, IFN-β and IFN-τ) which exhibit structural homology and are believed to be derived from the same ancestral gene, and Type II Interferon (IFN-gamma) which does not exhibit homology with the other interferons, but shares some biological activities. Both types of interferons enhance the expression of MHC molecules, which augment the cytolytic activity of T cells, thus making interferons useful T-cell enhancing agents. Interferons also activate natural killer (NK) cells, and macrophages, both of which become more effective at killing target cells. Numerous patents describe the production and use of various interferons. For example, U.S. Pat. No. 5,540,923 describes methods for isolating both Type I and Type II interferons and U.S. Pat. Nos. 5,376,567 and 4,889,803 relate to the recombinant expression of IFN-gamma. A form of IFN-gamma 1b known as Actimmune™ is manufactured by InterMune, Palo Alto, Calif. Low-doses of IFN-α have been used in treating chronic myeloid leukemia (Schofield et al., Ann. Intern. Med. 121:736; 1994) and other forms of cancer. A recombinant from of IFN-α, Introna®, is marketed by Schering-Plough for various anti-viral and anti-cancer indications.

Other agents that act on the various members of the TNF receptor superfamily of proteins will also have utility herein. Exemplary agents include agonistic antibodies, including humanized or single chain versions thereof. For example, Melero et al. have shown that monoclonal antibodies to 4-1BB can lead to the eradication of large, poorly immunogenic tumors in mice (Nature Med. 3:682; 1997). According to Melero et al., agonistic 4-1BB antibodies augment tumor-specific CTL activity. Accordingly, such antibodies (or 4-1BB ligands) may have use in the inventive method for upregulating CTL activity; they may also function to increase the amount of tumor antigen available by causing tumor cell death. U.S. Pat. No. 5,674,704, issued Oct. 7, 1997, discloses a ligand for 4-1BB that comprises a cytoplasmic domain, a transmembrane region and an extracellular domain. A soluble form of 4-1BB ligand comprising the extracellular domain is also disclosed; additional, multimeric forms are prepared by adding a multimer-forming peptide (such as an Fc molecule or a zipper peptide) to the extracellular domain. A particularly useful agonistic monoclonal antibody is 4-1BBm6 (deposited at the American Type Tissue Collection in Manassas, Va. on ______ and given accession number ______). Other forms of antibodies that bind the same epitope as 4-1BBm6 will also be useful, including humanized forms of murine antibodies, single chain antibodies, and monoclonal antibodies that are generated in transgenic mice that exhibit human antibody genes and therefor make human antibodies to antigens.

Similarly, agonists of OX40 (molecules that bind OX40 and transduce a signal thereby, including agonistic antibodies and OX40 ligand) promote a CD8+T cell response that can lead to the rejection of tumors. U.S. Pat. No. 5,457,035, issued Oct. 10, 1995, discloses a ligand for OX40; Miura et al. (Mol. Cell Biol. 11:1313; 1991) disclose a human homolog of murine OX40L which they refer to as gp34. Like other members of the TNF superfamily, OX40L is a type II transmembrane protein; soluble forms of OX40L are made from the extracellular domain. Multimeric forms of OX40L are prepared using standard recombinant DNA techniques to append a multimer-forming peptide such as an immunoglobulin Fc or an oligomerizing zipper to DNA encoding OX40L. A preferred agonistic monoclonal antibody is Ox40 m5 (deposited at the American Type Tissue Collection in Manassas, Va. on and given accession number). Other forms of antibodies that bind the same epitope as Ox40 m5 will also be useful, including humanized forms of murine antibodies, single chain antibodies, and monoclonal antibodies that are generated in transgenic mice that exhibit human antibody genes and therefor make human antibodies to antigens.

Those of skill in the art are also aware of a number of other factors that influence T cells, including Transforming Growth Factor-β (TGF-β). This cytokine can enhance the growth of immature lymphocytes, inhibits the apoptosis of T cells, and has a potent immunosuppressive effect on lymphocytes. Thus, TGF-β or inhibitors thereof (such as antibodies that bind TGF-β and prevent binding to cell-associated TGF-β receptor, soluble forms of TGF-β receptors, or other molecules that interfere with the ability of TGF-β to bind its receptor or transduce a signal thereby) will also be useful in the instant invention. The skilled artisan will be able to select appropriate forms to use, depending on the desired effects, by the application of routine experimentation.

Other molecules are also known to be crucial in the development of an immune response, and appear to preferentially enhance an immune response that is Tₕ2-like (that is, dominated by antibody-producing cells with little or no generation of cytotoxic T cells), including Interleukins 4, 5 and 10. Antagonists of these molecules will be useful in preventing or decreasing a Tₕ2-like immune response; in combination with the other aspects of the present invention, such antagonists facilitate the manipulation of an immune response toward a Tₕ1-like response, which may be more effective at eliminating target cells in an individual. Antagonists include antibodies that bind one of these molecules and prevent binding to cell-associated receptors therefor, soluble forms of receptors, or other molecules that interfere with the ability of the molecule to bind its receptor or transduce a signal thereby. U.S. Pat. No. 5,599,905, issued Feb. 4, 1997, discloses useful forms of soluble IL-4 receptor.

Chemokines are small, basic proteins that exhibit chemotactic activity for various types of immune system cells. The members of this family of proteins can be divided into roughly four groups based on the formation of disulphide bonds between cysteine residues and the presence or absence of intervening amino acids between the cysteine residues, which correlate approximately with function. Thus, members of the CXC subgroup exhibit an intervening amino acid between the first two hallmark cysteine residues, and tend to mainly attract and activate neutrophils. CC chemokines do not have an intervening amino acid, and exhibit chemotactic activity for dendritic cells, lymphocytes and mononuclear cells. The third subclass of chemokines is the C family, which lacks two of the four cysteines; it is represented by lymphotactin, a lymphoid-specific attractant that has been shown to attract NK and CD4 T cells to selected sites. A fourth type of chemokine with three intervening amino acids (CX3C) has also been identified; the representative molecule of this subfamily, fractalkine, may be involved in leukocyte adhesion and extravasation.

Accordingly, chemokines will find use in the instant invention to attract particular types of cells to a particular site (i.e., a site of infection). For example, a CC chemokine such as one of MCPs 1-5, MIP-1 alpha or beta, RANTES or eotaxin, may be given locally at the site of infection by any of the techniques known in the art and discussed herein (i.e., by intra-nodal injection of the protein or DNA encoding it, or through use of a gene therapy technique to induce secretion of the chemokine by cells at the site), to attract mobilized dendritic cells to the site. The chemokine used can be selected, depending on the type of cell to be attracted, by the application of routine experimentation.

Additional useful agents are disclosed in U.S. Ser. No. 60/249,524, filed Nov. 17, 2000, the disclosure of which is incorporated by reference herein. In particular, the chemokines MIP-3alpha, MIP-3beta, MIP-5, MDC, SDF-1, MCP-3, MCP4, RANTES, TECK, and SDF-1 are useful chemokines that act as dendritic cell localization factors. Moreover, cytokines such as IL-1, TNF-alpha and IL-10 are also capable of acting as localization factors. Compounds that bind to and activate one or more members of the somatostatin cell surface receptors SSTR1, SSTR2, SSTR3, SSTR4 and SSTR5 or homologs or orthologs thereof will also be useful in the inventive methods. These include the naturally occurring ligands for the somatostatin receptors, including somatostatin and cortistatin, and somatostatin peptides SST-14, SST-28 and cortistatin peptides CST-17 and CST-29. Other known peptide agonists of SSTRs include ocreotide, lanreotide, vapreotide, seglitide, BM23268, NC8-12, BIM23197, CD275 and other found to have high affinity for SSTRs. Derivatives, analogs and mimetics of any of these compounds will also be useful in the present invention.

It is understood by those of skill in the art that the various agents and/or factors disclosed herein act by binding to cell surface receptors and transducing a signal to the cell thereby. It is also understood that other agents can also exhibit these characteristics (i.e., agonistic antibodies to a given receptor). Accordingly, the inventive methods encompass the use of other molecules that mimic the signaling to cells that occurs with the factors that are specifically disclosed above. Such molecules include agonistic monoclonal antibodies and recombinant proteins derived therefrom as well as ligand mimetics isolated by screening small molecule libraries or through rational drug design.

In Vitro and In Vivo Models

Those of skill in the art routinely use animal models and/or in vitro systems for testing therapeutic agents in the infectious disease setting. For example, Sher (Imm. Rev. 127:183-204, 1992), discusses murine models of several different human diseases, including acquired immunodeficiency syndrome (AIDS), toxoplasmosis, leishmaniasis, trypanosomiasis, and shistosomiasis. Nathan (in: Mechanisms of Host Resistance to Infectious Agents, Tumors, and Allografts, R. M. Steinman and R. J. North, eds., Rockefeller University Press, New York, pp.165-184, 1986) also reviews the use of mice in the study of various human diseases, and further presents results of studies performed in humans that confirm results first observed in murine models. Rats and/or mice have also been used in animal models of cryptosporidiosis (Meulbroek et al., Workshop on Pneumocystis, Cryptospridium and Microsporidium 113S), Salmonella typhimurium infections (Hougen et al., APMIS 98:30; 1990), Mycobacterium avium infections (Furney et al., Antimicrobial Agents and Chemotherapy 34:1629; 1990), and of Pneumocystis carini pneumonia (Boylan and Current, J. Protozool. 38:138S; 1991; Soulez et al., Workshop on Pneumocystis, Cryptospridium and Microsporidium 123S)

Other species also provide useful animal models. For example, Wyand (AIDS Res. and Human Retroviruses 8:349; 1992) discusses the use of SIV-infected Rhesus monkeys for the preclinical evaluation of AIDS drugs and vaccines. Simian and feline models (Gardner, Antiviral Res. 15:267; 1991; Stahl-Hennig et al., AIDS 4:611; 1990) and murine models (Ruprecht et al., Cancer Res. 50:5618s; 1990) have been proposed for evaluating anti-retroviral therapy. Rhesus monkeys have also been used in a model of Chagas' disease (Bonecini-Almeida et al., Mem. Inst. Osaldo Cruz 85:163; 1990; Rio de Janeiro). Various non-human primates have been observed to suffer naturally-acquired or experimentally-acquired leprosy (Meyers et al., Am. J. Trop. Med and Hyg. 44:24; 1991). Those skilled in the art recognize these and many other possible animal models of disease useful in evaluating therapeutic methods and determining therapeutically effective dosages.

Administration of Immunomodulatory Agents

The present invention provides methods of using therapeutic compositions comprising one or more immunomodulatory agents and a suitable diluent and carrier, and methods for regulating an immune or inflammatory response. The use of various combinations of immunomodulatory agents is also contemplated. For example, Flt3L can be used in conjunction with factors that are known to enhance a CTL response, such as OX40 and/or 4-1BB agonists.

For therapeutic use, an immunomodulatory agent or combination thereof is administered to a patient, preferably a human, for treatment in a manner appropriate to the indication. Thus, for example, immunomodulatory agents administered to augment immune and/or inflammatory response can be given by bolus injection, continuous infusion, sustained release from implants, repeated daily (or periodic) injections for a preferred period of time, or other suitable technique. Typically, such agents will be administered in the form of a pharmaceutical composition comprising purified compound or combination thereof in conjunction with physiologically acceptable carriers, excipients or diluents. Such carriers will be nontoxic (or mininially toxic) to recipients at the dosages and concentrations employed.

Ordinarily, the preparation of such compositions entails combining a compound with buffers, antioxidants such as ascorbic acid, low molecular weight (less than about 10 residues) polypeptides, proteins, amino acids, carbohydrates including glucose, sucrose or dextrans, chelating agents such as EDTA, glutathione and other stabilizers and excipients. Neutral buffered saline or saline mixed with conspecific serum albumin are exemplary appropriate diluents. Pharmaceutical compositions may be formulated as a lyophilizate using appropriate excipient solutions (e.g., sucrose) as diluents.

Moreover, various means of achieving controlled or sustained release of pharmaceutical compositions are known in the art. For example, U.S. Pat. No. 5,942,253 discloses prolonged-release compositions comprising GM-CSF. Other types of controlled release technology are known in the art (for example, the use of hydrogels as disclosed herein), and can be prepared by those of ordinary skill in the for use in the instant invention. The particular therapeutic effective amount employed is not critical to the present invention, and will vary depending upon the particular factor selected, the disease or condition to be treated, as well as the age, weight and sex of the individual.

The DC mobilization factors, DC maturation factors, and CTL enhancing factors may be administered in a suitable diluent or carrier to an individual, preferably a human. Thus, for example, any one or all of these factors can be given by bolus injection, continuous infusion, sustained release from implants, or other suitable technique. Moreover, the factors can be administered by using gene therapy techniques. For example, DC can be transfected with a gene encoding a CTL enhancing factor such as IL-2, IL-12, or IL-15. The transfected DC are administered to the individual to provide a stronger and improved immune response to an antigen. Those of skill in the art will be able to perform routine experimentation using animal models or other modeling systems to determine preferable routes of administration and amounts of various factors to deliver (see, for example, the discussion in U.S. Pat. No. 6,017,540, issued Jan. 25, 2000, relating to dosage calculations and animal models).

The particular therapeutically effective amount employed is not critical to the present invention, and will vary depending upon the particular factor selected, the disease or condition to be treated, as well as the age, weight and sex of the individual. Additionally, the time at which a given factor is given will depend on the individual factor administered and its activity. Typical therapeutically effective dosages of various factors and typical intervals at which to administer them are shown in Table 1 below. Those of ordinary skill in the art are able to optimize dosages and routes of administration of these and other factors by the application of routine experimentation.

TABLE 1
Typical Therapeutic Dosages

| Factor | Dosage Range | Administer at: |
|---|---|---|
| FIt3L | 10-100 | for 10 to 25 days; daily or |
| | μg/Kg | every other day; or via slow |
| | | or controlled release |
| GM-CSF | 100-300 | 10 to 25 days; daily or every |
| | μg/Kg | other day; or via slow or |
| | | controlled release |
| IL-15 | 10 μg/Kg- | 24 to 48 hours after |
| | 10 mg/Kg | administration of DC |
| | | mobilization factors, to |
| | | stimulate NK and/or |
| | | activation of CTL |
| CD40L | 10 to 200 | During the last 7-10 days |
| | μg/Kg | of treatment with DC |
| | | mobilization factors, to |
| | | stimulate maturation of DC |
| | | and/or activation of CTL or |
| | | when the number of DCs |
| | | peaks |
| RANKL | 10 to 200 | 24 to 48 hours after |
| | μg/Kg | administration of DC |
| | | mobilization factors, to |
| | | stimulate maturation of DC |
| | | and/or activation of CTL or |
| | | when the number of DCs |
| | | peaks |
| OX40 | 10 μg/Kg- | 2-4 injections spaced |
| agonists | 10 mg/Kg | approximately 3 days apart, |
| | | beginning when peak DC |
| | | mobilization occurs. |
| 4-1BB | 10 μg/Kg- | 2-4 injections spaced |
| agonists | 10 mg/Kg | approximately 3 days apart, |
| | | beginning when peak DC |
| | | mobilization occurs. |

Administration of a DC mobilization or maturation factor or CTL enhancing factor as a local agent may allow the use of agents that are not desirable for systemic use (for example, TNF), and may be used to achieve higher concentrations of various agents at a particular site than could safely be achieved using systemic administration. Similarly, agents that act as attractants for DC or CTL will also be useful for administration in or near a specific site. Such local administration allows concentration of effector cells at the site while minimizing systemic effects.

Various means may be used to achieve localized administration, including local injection of protein, use of gene therapy techniques to induce expression of recombinant protein in or near the site of infection, and use of site-specific and/or controlled release technology. Moreover, it has been found that raw DNA, when injected into a mammal, is often taken up by cells and expressed. Accordingly, DNA encoding a desired factor may be injected into or near the site, and, when taken up by nearby cells, will result in the localized expression of the factor encoded thereby.

One type of technology that may be useful for localized administration is that utilizing hydrogel materials to achieve sustained release of a desired factor or factors, for example, photopolymerizable hydrogels (Sawhney et al., Macromolecules 26:581; 1993). Similar hydrogels have been used to prevent postsurgical adhesion formation (Hill-West et al., Obstet. Gynecol. 83:59; 1994) and to prevent thrombosis and vessel narrowing following vascular injury (Hill-West et al., Proc. Natl. Acad. Sci. USA 91:5967; 1994). Proteins can be incorporated into such hydrogels to provide sustained, localized release of active agents (West and Hubbell, Reactive Polymers 25:139; 1995; Hill-West et al., J. Surg. Res. 58:759; 1995).

Accordingly, the various factors disclosed herein can also be incorporated into hydrogels, for application to tissues for which localized administration is desirable. For example, a hydrogel incorporating a DC attractant, DC maturational factor, or CTL enhancing factor, or a combination of various such factors, can be applied to tissue after surgery. Moreover, such hydrogel-based formulations may be administered by other methods that are known in the art, for example using a catheter to apply the hydrogel at a desired location in the vascular system, or by any other means by which local administration can be accomplished. Those of ordinary skill in the art will be able to formulate an appropriate hydrogel by applying standard pharmacokinetic studies, for example as discussed by West and Hubbell, supra.

Ex Vivo Culture of DC and/or CTL

Those of skill in the art will also recognize that various ex vivo culture techniques can also be employed in the present invention. A procedure for ex vivo expansion of hematopoietic stem and progenitor cells is described in U.S. Pat. No. 5,199,942, incorporated herein by reference. U.S. Pat. No. 6,017,527 describes a method of culturing and activating DC; other suitable methods are known in the art. In one aspect of the invention, ex vivo culture and expansion comprises: (1) collecting CD34+hematopoietic stem and progenitor cells from a patient from peripheral blood harvest or bone marrow explants; and (2) expanding such cells ex vivo. In addition to the cellular growth factors described in U.S. Pat. No. 5,199,942, other factors such as Flt3L, IL-1, IL-3, RANKL and c-kit ligand, can be used.

Stem or progenitor cells having the CD34 marker constitute only about 1% to 3% of the mononuclear cells in the bone marrow. The amount of CD34⁺ stem or progenitor cells in the peripheral blood is approximately 10- to 100-fold less than in bone marrow. In the instant invention, cytokines such as Flt3L, GM-CSF, CD40L and IL-15 may be used to increase or mobilize the numbers of stem cells to the peripheral blood in vivo. Such cells are then obtained and cultured using methods that are known in the art (see, for example, U.S. Pat. Nos. 5,199,942, and 6,017,527).

Isolated stem cells can be frozen in a controlled rate freezer (e.g., Cryo-Med, Mt. Clemens, MI), then stored in the vapor phase of liquid nitrogen using dimethylsulfoxide as a cryoprotectant. A variety of growth and culture media can be used for the growth and culture of dendritic cells (fresh or frozen), including serum-depleted or serum-based media. Useful growth media include RPMI, TC 199, Iscoves modified Dulbecco's medium (Iscove, et al., F. J. Exp. Med., 147:923 (1978)), DMEM, Fischer's, alpha medium, NCTC, F-10, Leibovitz's L-15, MEM, AIM-V and McCoy's.

The collected CD34⁺ cells are cultured with suitable cytokines, for example, as described herein, and in the aforementioned patents. CD34⁺ cells then are allowed to differentiate and commit to cells of the dendritic lineage. These cells are then further purified by flow cytometry or similar means, using markers characteristic of dendritic cells, such as CD1a, HLA DR, CD80 and/or CD86. Purified dendritic cells may pulsed with (exposed to) a desired antigen (for example, a purified antigen that is specific for the organism at issue, a crude antigen preparation or DNA or RNA encoding a particular antigen or antigens), to allow them to take up the antigen in a manner suitable for presentation to other cells of the immune systems.

Antigens are classically processed and presented through two pathways. Peptides derived from proteins in the cytosolic compartment are presented in the context of Class I MHC molecules, whereas peptides derived from proteins that are found in the endocytic pathway are presented in the context of Class II MHC. However, those of skill in the art recognize that there are exceptions; for example, the response of CD8⁺ antigen specific T cells, which recognize antigens expressed on MHC Class I. A review of MHC-dependent antigen processing and peptide presentation is found in Germain, R. N., Cell 76:287 (1994).

Numerous methods of pulsing dendritic cells with antigen are known; those of skill in the art regard development of suitable methods for a selected antigen as routine experimentation. In general, the antigen is added to cultured dendritic cells under conditions promoting viability of the cells, and the cells are then allowed sufficient time to take up and process the antigen, and express or present antigen peptides on the cell surface in association with either Class I or Class II MHC, a period of about 24 hours (from about 18 to about 30 hours, preferably 24 hours). Dendritic cells may also be exposed to antigen by transfecting them with DNA encoding the antigen. The DNA is expressed, and the antigen is presumably processed via the cytosolic/Class I pathway. Additionally, DC can be induced to present antigen by contacting them with mRNA amplified from the organism(s).

After antigen has been processed, the DC are contacted with a DC maturation factor such as CD40L. CD40L and other DC maturation factors increase the numbers of MHC molecules (and costimulatory molecules such as CD80 and CD86 as well as Ox40L) on the surface of the DC, thereby enhancing their antigen-presenting ability. Moreover, DC that have been exposed to maturation factors secrete cytokines that are indicative of activation (for example, IL-12, IL-15). CD4+ cells that are presented antigen by mature, activated DC will express IL-2, IL4, and IFN-gamma, which act as growth factors for T cells. Accordingly, mature, activated DC are able to stimulate an effective, antigen-specific immune response.

Smaller antigens such as peptides do not require processing by the dendritic cell, but are bound to the appropriate MHC molecules upon exposure of the DC to the peptides. When a peptide antigen is used, it is advantageous to stimulate the maturation of the DC prior to (or concurrently with) exposure to the peptide antigen, in order to increase the numbers of available MHC molecules, and thereby enhance antigen-carrying capacity. The same DC maturation factors that are useful in stimulating the maturation of DC that have processed larger protein antigens will also be useful in augmenting the capacity of DC to present smaller peptide antigens.

The activated, antigen-carrying DC are then administered to an individual in order to stimulate an antigen-specific immune response. The DC may be administered systemically, or they may be administered locally into or near a selected site. If it is desired, additional agents such as T cell enhancing factors can be administered to the individual to further enhance the immune response. The DC can be administered prior to, concurrently with, or subsequent to, administration of additional agents. Alternatively, T cells may be collected from the individual and exposed to the activated, antigen-carrying dendritic cells in vitro to stimulate development of antigen-specific T cells ex vivo, which are then administered to the individual.

Administration of Activated, Antigen-pulsed Dendritic Cells

The present invention provides methods of using therapeutic compositions comprising activated, antigen-pulsed dendritic cells. The use of such cells in conjunction with soluble cytokine receptors or cytokines, or other immunoregulatory molecules is also contemplated. The inventive compositions are administered to stimulate an immune response, and can be given by bolus injection, continuous infusion, sustained release from implants, or other suitable technique. Typically, the cells of the inventive methods will be administered in the form of a composition comprising the antigen-pulsed, activated dendritic cells in conjunction with physiologically acceptable carriers, excipients or diluents. Such carriers will be nontoxic to recipients at the dosages and concentrations employed. Neutral buffered saline or saline mixed with conspecific serum albumin are exemplary appropriate diluents.

For use in stimulating a certain type of immune response, administration of other cytokines along with activated, antigen-pulsed dendritic cells is also contemplated. Several useful cytokines (or peptide regulatory factors) are discussed in Schrader, J. W. (Mol Imniunol 28:295; 1991), and in are described in The Cytokine Handbook (third edition; edited by Angus Thompson; Academic Press 1998). Such factors include (alone or in combination) Interleukins 1, 2, 4, 5, 6, 7, 10, 12 and 15; granulocyte-macrophage colony stimulating factor, granulocyte colony stimulating factor; a fusion protein comprising Interleukin-3 and granulocyte-macrophage colony stimulating factor; Interferon-γ; TNF, TGF-β (or inhibitors thereof), flt-3 ligand and biologically active derivatives thereof.

The additional factors or agents are administered to the individual to further enhance the immune response. The dendritic cells can be administered prior to, concurrently with, or subsequent to, administration of additional agents. Alternatively, T cells may be collected from the individual and exposed to the activated, antigen-carrying dendritic cells in vitro to stimulate development of antigen-specific T cells ex vivo, which are then administered to the individual.

Prevention or Treatment of Disease

These results presented herein indicate that combination therapy may be of significant clinical use in the treatment of various diseases. The term treatment, as it is generally understood in the art, refers to initiation of therapy after clinical symptoms or signs of disease have been observed. Accordingly, the inventive methods may be used to treat an individual who is manifesting signs or symptoms of disease. Moreover, the inventive methods may be used in individuals who do not exhibit signs or symptoms, but who are at risk for a disease or diseases. In such uses, the present methods are often thought of as preventative or prophylactic measures that function to reduce the likelihood that an individual at risk for contracting a disease will actually succumb to the disease. Accordingly, the inventive methods will also be useful in immunization regimens.

The relevant disclosures of all publications cited herein are specifically incorporated by reference. The following examples are intended to illustrate particular embodiments, and not limit the scope, of the invention. Those of ordinary skill in the art will readily recognize that additional embodiment are encompassed by the invention.

### EXAMPLE 1

This example illustrates the use of an Ox40 agonist in enhancing an immune response to an antigen. Splenic dendritic cells (DC) are obtained from BALB/c mice, separated into CD8+ and CD8−subsets, pulsed in vitro with a protein antigen (keyhole limpet hemocyanin or KLH), and administered to naive BALB/c mice. Administration of antigen-pulsed CD8−DC lead to the development of a mixed Th response, as determined by observing the secretion of IL-2, [IL-4, IL-5, IL-10 and IFN-gamma by T cells obtained from the mice. In contrast, administration of CD8+DC resulted in the development of a Tₕ1-like response, with the generation of T cells that secreted IL-2 and IFN-gamma. When an OX40 agonist (monoclonal antibody OX40M5) is administered along with antigen-pulsed DC of either subtype, the resultant immune response is primarily Tₕ1-like, with high levels of IFN-gamma-secreting cells.

### EXAMPLE 2

This example demonstrates that the use of a CD40 agonist results in the expression of OX40 ligand (OX40L) by DC. Splenic DC, which do not express OX40 when freshly isolated, are obtained from BALB/c mice, and contacted with a CD40 agonist (a soluble form of CD40L referred to as trimeric CD40L). Beginning at approximately 48 hours after CD40L-stimulation, the DC express cell-surface associated OX40L, as determined by fluorescence-activated cell sorting using an OX40/Fc fusion protein.

The OX40L-expressing DC are pulsed in vitro with an antigen, and administered to naive BALB/c mice. Administration of antigen-pulsed, OX40L-expressing DC lead to the development of a predominantly Tₕ1-like response, with the generation of T cells that secreted IL-2 and IFN-gamma. When an OX40 agonist (monoclonal antibody OX40M5) is administered along with antigen-pulsed DC, the resultant immune response is primarily Tₕ1-like, with high levels of IFN-gamma-secreting cells.

### EXAMPLE 3

This example illustrates the ability of OX40 agonist Ox40 m5 to increase CD8 T cell activation induced by dendritic cells. A small but detectable number of naive cells from OVA-specific CD8 transgenic mice (OT.I) was transferred intravenously into naive recipients. One day after transfer, the animals were immunized subcutaneously in the hind footpads with 3×10⁵ mature dendritic cells (from Flt3L treated wild-type or MHC Class II knockout animals) pulsed with the class I OVA peptide. On the same day, the animals were also injected intraperitoneally with Ox40 m5 (100 μg) or a control monoclonal antibody. T cell expansion in the draining lymph node was monitored by FACS five days after immunization.

Co-injection of Ox40 m5 and OVA peptide-pulsed wild type dendritic cells (but not dendritic cells from class I knockout mice) strongly enhanced the CD8 T cell expansion. Lymph node cells from these immunized animals were also restimulated in vitro with the antigen. The supernatants from these cultures were assessed for IFN-gamma production. Co-immunization with wild-type dendritic cells and Ox40 m5 enhanced production of IFN-gamma as compared to immunization with dendritic cells alone. Lymph node cells from mice immunized with class I knockout dendritic cells produced low levels of IFN-gamma upon restimulation in vitro, and the co-injection of Ox40 m5 did not enhance this production. These data suggest that OX40 agonists enhance CD8 T cell expansion and activation in vivo, and thus enhance an antigen-specific effector T cell response.

### EXAMPLE 4

This example describes the effects of antibody Ox40 m5 with or without Flt3L on the ability of mice to reject a challenge of fibrosarcoma cell in a murine model of fibrosarcoma substantially as described in Lynch et al., Eur. J. Immunol. 21:1403 (1991). Six to eight week old C57BL/10J (B10) mice were inoculated with about 1×10⁵ B10 fibrosarcoma cells subcutaneously in the foot. Therapy with either Flt3L (10 μg per mouse intraperitoneally on each of days 10 through 29), Ox40 m5 (10 μg per mouse intraperitoneally every third day from days 10 through 27), or both, was initiated ten days after inoculation. All control mice developed tumors, as did 80% of mice given Flt3L alone, whereas 30% of mice treated with Ox40 m5 and 50% of mice treated with Ox40 m5 plus Flt3L rejected their tumors.

A similar experiment was done with another fibrosarcoma, referred to as 87, in C3H mice, utilizing two different doses of Ox40 m5 (either 100 μg per mouse or 500 μg per mouse), given on days 5, 9, 11 and 13. With the higher dose (500 μg per mouse), 40% of mice rejected the tumors, while 30% of the mice given the lower dose rejected their tumors. When Ox40 m5 was given in combination with 4-1BBm6 using substantially the same parameters, 100% of the mice given both antibodies rejected the tumor, while 60% that received 4-1BBm6 alone rejected tumor challenge.

The combination of Ox40 m5 and 4-1BBm6 was also investigated in a renal cell carcinoma model. This combination, alone or with the addition of Flt3L, did not yield significant protection from tumor challenge (only 10% of mice rejected tumor challenge), however, tumor growth was slower in animals treated with either Ox40 m5 and 4-1BBm6, or Ox40 m5,4-1BBm6 and Flt3L. The renal carcinoma cell used are known to generate a rapidly growing tumor; accordingly, the combination of Ox40 m5 and 4-1BBm6 may prove useful even when the tumor is known to be very aggressive if given in combination with other therapy that affects the growth of the tumor.

Because rejection of tumor cells is dependent mostly upon a Tₕ1-like immune response, these results indicate that the combination of an OX40 agonist and a 4-1BB agonist will enhance a Tₕ1 response, and would be expected to be effective in the treatment or prevention of infectious disease for which a Tₕ1 immune response is desirable.

What is claimed is:

1. A method for treating an individual at risk for or suffering from infection with a pathogenic or opportunistic organism, comprising the steps of:
(a) administering a therapeutically effective amount of a dendritic cell mobilization factor to the individual; and
(b) administering a therapeutically effective amount of a dendritic cell maturation agent to the individual.

2. The method of claim 1, wherein the dendritic cell mobilization factor is Flt3L, and the dendritic cell maturation agent is CD40L.

3. A method for treating an individual at risk for or suffering from infection with a pathogenic or opportunistic organism comprising the steps of:
(a) administering a therapeutically effective amount of a dendritic cell mobilization factor to the individual;
(b) administering a therapeutically effective amount of a dendritic cell maturation agent to the individual; and
(c) administering a therapeutically effective amount of a dendritic cell activation agent to the individual.

4. The method of claim 3, wherein the dendritic cell mobilization factor is Flt3L, and the dendritic cell maturation agent is CD40L.

5. The method of any one of claims 1 through 4, wherein a T cell enhancing factor is administered in conjunction with the dendritic cell maturation agent.

6. The method of claim 5, wherein the T cell enhancing factor is Interleukin-15.

7. The method of claim 5, wherein the T cell enhancing factor is selected from the group consisting of:
(a) Ox40 agonists;
(b) 4-1BB agonists; and
(c) combinations of Ox40 agonists and 4-1BB agonists.

8. A method for treating an individual at risk for or suffering from infection with a pathogenic or opportunistic organism comprising the steps of:
(a) administering a therapeutically effective amount of a dendritic cell mobilization factor to the individual;
(b) obtaining dendritic cells from the individual and culturing the dendritic cells ex vivo;
(c) administering the dendritic cells to the individual; and
(d) administering a T cell enhancing factor to the individual.

9. The method of claim 8, wherein the dendritic cells are contacted with a dendritic cell maturation agent ex vivo.

10. The method of claim 9 wherein the dendritic cells are contacted with an antigen prior to being contacted with the dendritic cell maturation agent.

11. The method of claim 9 wherein the dendritic cells are contacted with an antigen after being contacted with the dendritic cell maturation agent.

12. The method of any one of claims 8 through 11, wherein the dendritic cell mobilization factor is Flt3L, and the dendritic cell maturation agent is CD40L.

13. The method of any one of claims 8 through 11, wherein the T cell enhancing factor is selected from the group consisting of:
(a) Ox 40 agonists;
(b) 4-1BB agonists;
(c) combinations of Ox40 agonists and 4-1BB agonists; and
(d) Interleukin-15.

14. A method for treating an individual at risk for or suffering from infection with a pathogenic or opportunistic organism comprising the steps of:
(e) administering a therapeutically effective amount of a dendritic cell mobilization factor to the individual;
(f) obtaining dendritic cells from the individual and culturing the dendritic cells ex vivo;
(g) causing the dendritic cells to become mature and active and express antigen;
(h) obtaining T cells from the individual;
(i) contacting the T cells ex vivo with the mature, active, antigen-expressing dendritic cells to obtain activated, antigen-specific T cells; and
(j) administering the activated, antigen-specific T cells to the individual.

15. The method of claim 14 wherein a T cell enhancing agent is administered to the individual before the T cells are obtained from the individual.

16. The method of claim 14 or claim 15 wherein a T cell enhancing agent is administered to the individual in conjunction with the activated, antigen-specific T cells.

17. The method of claim 16, wherein the T cell enhancing factor is selected from the group consisting of:
(a) Ox 40 agonists;
(b) 4-1BB agonists;
(c) combinations of Ox40 agonists and 4-1BB agonists; and
(d) Interleukin-15.

18. The method of anyone of claims 1 through 17, wherein a T cell attractant is administered to attract T cells to a specific site.

19. The method of anyone of claims 1 through 18, wherein a dendritic cell attractant is administered to attract dendritic cells to a specific site.


SEQUENCE LISTING (6 sequences)

SEQ ID NO: 1 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - protein, 33 aa, Artificial Sequence
  Zipper peptide
RMKQIEDKIEEILSKIYHIENEIARIKKLIGER

SEQ ID NO: 5 - protein, 281 aa, Homo sapiens [sequence omitted]

SEQ ID NO: 6 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID